(19) United States
(12) Patent Application Publication
(10) Pub. No.: US 2023/0048025 A1
(43) Pub. Date: Feb. 16, 2023

(54) ADENO ASSOCIATED VIRAL VECTORS

(71) Applicant: OREGON HEALTH & SCIENCE UNIVERSITY, Portland, OR (US)

(72) Inventors: Hiroyuki Nakai, Portland, OR (US); Kei Adachi, Portland, OR (US)

(21) Appl. No.: 16/472,793

(22) PCT Filed: Dec. 21, 2017
(86) PCT No.: PCT/US2017/068050

§ 371 Date: Jun. 21, 2019

Related U.S. Application Data
(60) Provisional application No. 62/438,255, filed on Dec. 22, 2016.

(51) Int. Cl. C12N 15/86; C07K 14/005

(52) U.S. Cl. CPC C12N 15/86; C07K 14/005; C12N 2750/14143; C12N 2750/14122; C12N 2750/14171

(57) ABSTRACT

Recombinant adeno associated viral vectors are disclosed. These include AAV8 or AAV9 derived vectors that include capsid proteins with mutations that confer upon the vector particular characteristics such as the ability to transduce or avoid (detarget) particular tissues, to be retained longer in the blood, or to be internalized in a cell without viral expression.

### ACKNOWLEDGEMENT OF GOVERNMENT SUPPORT

This invention was made with the support of the United States government under the terms of grant number NS088399 awarded by the National Institutes of Health. The United States government has certain rights to this invention.

### FIELD

Generally, the field involves adeno associated viruses with engineered mutations in the capsid domain. More particularly, the field involves adeno associated viruses that target or avoid particular tissues.

### BACKGROUND

Recombinant adeno-associated virus (rAAV) is among the most promising vectors for in vivo gene delivery. The usefulness of rAAV vectors has been expanded since a number of naturally occurring new serotypes and subtypes were isolated from human and non-human primate tissues (Gao G et al, J Virol 78, 6381-6388 (2004) and Gao G P et al, Proc Natl Acad Sci USA 99, 11854-11859 (2002); both of which are incorporated by reference herein). Among the newly-identified AAV isolates, AAV serotype 8 (AAV8) and AAV serotype 9 (AAV9) have gained attention because rAAV vectors derived from these two serotypes can transduce various organs including the liver, heart, skeletal muscles and central nervous system with high efficiency following systemic administration (Ghosh A et al, Mol Ther 15, 750-755 (2007); Pacak C A et al, Circ Res 99, 3-9 (2006); Inagaki K et al, Mol Ther 14, 45-53 (2006); Zhu T et al, Circulation 112, 2650-2659 (2005); Wang Z et al, Nat Biotechnol 23, 321-328 (2005); Nakai H et al, J Virol 79, 214-224 (2005); and Foust K D et al, Nature Biotechnol 23, 321-328 (2009); all of which are incorporated by reference herein). This robust transduction by rAAV8 and 9 vectors has been presumed to be ascribed to strong tropism for these cell types, efficient cellular uptake of vectors, and/or rapid uncoating of virion shells in cells (Thomas C E et al, J Virol 78, 3110-3122 (2004); incorporated by reference herein). In addition, emergence of capsid-engineered rAAV with better performance has significantly broadened the utility of rAAV as a vector toolkit (Asokan A et al, Mol Ther 20, 699-708 (2012); incorporated by reference herein).

A proof-of-concept using rAAV-mediated gene therapy has been shown in many preclinical animal models of human diseases. Phase I/II clinical studies have shown promising results for the treatment for hemophilia B (Nathwani A C et al, N Engl J Med 71, 1994-2004 (2014); incorporated by reference herein); lipoprotein lipase deficiency (Carpentier A C et al, J Clin Endocrinol Metab 97, 1635-1644 (2012); incorporated by reference herein); Leber congenital amaurosis (Jacobson S G et al, Arch Ophthalmol 130, 9-24 (2012) and Pierce E A and Bennett J, Cold Spring Harb Perspect Med 5, a017285 (2015); both of which are incorporated by reference herein), among others (reviewed in Mingozzi F and High K A, Nat Rev Genet 12, 341-355 (2011); incorporated by reference herein). Despite this promise, human studies have also revealed unexpected issues and potential challenges in rAAV-mediated gene therapy (Manno C S et al, Nat Med 12, 342-347 (2006); incorporated by reference herein).

Although rAAV vectors have widely been used in preclinical animal studies and have been tested in clinical safety studies in many diseases, the current rAAV-mediated gene delivery systems face challenges, especially with regard to broader clinical applications, potentially due to a lack of understanding of the mechanisms of rAAV transduction in vivo. Mechanistic studies of the AAV capsid have been a significant challenge due to the multifunctional nature of the AAV viral capsid protein, mutants of which result in a wide array of phenotypes. Such phenotypes include efficiency of capsid assembly, ability to interact with AAV viral nonstructural proteins (i.e., Rep and AAP proteins), ability to interact with components in body fluid and extracellular matrix, blood clearance rates, vascular permeability, antigenicity, reactivity to neutralizing antibodies, tissue/organ/cell type tropism, efficiency of cell attachment and internalization, intracellular trafficking routes, virion uncoating rates, and so on. Even with information about the atomic structure of the AAV capsid, the relationships between mutations of the AAV capsid amino acid sequence and those of AAV phenotypes are difficult to predict. Many of these phenotypes have significant influences on the yield of rAAV vector production and the degree of efficiency in overcoming various barriers toward establishing infection/transduction; therefore, understanding of the capsid biology is imperative to improve the current system and develop novel vectors with desired properties.

### SUMMARY

Disclosed are AAV vectors comprising mutant capsid proteins with biological properties useful in clinical translation. Such vectors have particular characteristics such as detargeting the liver while transducing one or more tissues such as brain, heart, or skeletal muscle. Other such vectors detarget all of the brain, heart, lung, liver, kidney, spleen, intestine, pancreas, testis, muscle and fat. Such vectors can be engineered to have one or more additional capsid mutations that confer upon the vector the ability to transduce one or more of the above listed tissues or other tissues such as skin, glands, or other tissues. Other such vectors specifically target and transduce the heart but detarget all the brain, ling, liver, kidney, spleen, intestine, pancreas, testis, muscle and fat. Other such vectors are retained in the blood longer than vectors comprising an unmutated capsid. Other such vectors bind to cell surface galactose and are internalized. Some such vectors are internalized within the cell and their genomes degraded. Other such vectors are internalized within the cell and not degraded but do not express transgene products. Such vectors also include vectors of SEQ ID NO: 3-108 herein.

### BRIEF DESCRIPTION OF THE DRAWINGS

FIG. 1A is a graph showing transduction efficiencies of AAV9W22A/W23A in various tissues 6 weeks post injection determined by RNA Barcode-Seq. The transduction efficiency in each tissue is normalized by that of the wild-type AAV9. Error bars are mean±SEM (n=12).

FIG. 1B is an image of liver tissue and heart tissue eleven days post injection of the indicated constructs. Transduction efficiencies in the liver and heart were determined by X-Gal staining. Error bars are mean±SEM (n=3).

FIG. 1C is a graph showing double-stranded vector genome copy number for diploid equivalent (genomes per cell) in the heart as determined by quantitative PCR. Error bars are mean±SEM (n=3).

FIG. 1D is a graph showing double-stranded vector genome copy number for diploid equivalent (genomes per cell) in the liver as determined by quantitative PCR. Error bars are mean±SEM (n=3).

FIG. 1E is a graph showing transduction efficiencies in the heart determined by Xgal-staining of histological sections that are presented in FIG. 1B. Error bars are mean±SEM (n=3).

FIG. 2A is a graph showing transduction efficiencies of AAV9N272A in various tissues 6 weeks post injection determined by RNA Barcode-Seq. The transduction efficiency in each tissue is normalized by that of the wild-type AAV9. Error bars are mean±SEM (n=12).

FIG. 2B is a set of images showing liver-detargeting AAV9 mutants that transduce the brain at high levels. Double-stranded AAV-hSynl-GFP vector genome, packaged with 4 different capsids indicated in the figure (AAV9, AAV9N272A, AAV9-PHPB, AAV9N272A-PHPB) were injected into 8-week-old male C57BL/6 mice via the tail vein at a dose of 3x10e11 vg/mouse (n=2 or 3). AAV hSyn-GFP vector expresses GFP under the control of the human synapsin I gene enhancer-promoter. Eleven days post-injection, transduction efficiencies in neurons were determined by immunofluorescence microscopy using an anti-GFP antibody. Double-stranded vector genome copy numbers per diploid genomic equivalent (i.e., copy/cell) in the liver were determined by qPCR, and the results are shown in the figure. Values are mean±SD for triplicated samples or mean±|mean−each value| for duplicated samples.

FIG. 2C is a graph showing transduction efficiencies of various brain regions with the liver-detargeting AAV9 mutants that transduce the brain at high levels. The histological sections shown in FIG. 2B were assessed quantitatively in the following six brain regions (n=2 or 3): medial basal hypothalamus (MBH), cerebral cortex (CTX), hippocampus (Hc), caudate-putamen (CP), amygdala (Amyg) and thalamus (Thal). Error bars are SEM for triplicated samples or |mean−each value| for duplicated samples.

### SEQUENCE LISTING

SEQ ID NO: 1—AAV8 capsid:

SEQ ID NO: 2—AAV9 capsid

SEQ ID NO: 3-108 are mutant AAV8 or AAV9 vector capsids.

SEQ ID NO: 109 is a capsid from AAV-PHP.B with an N272A mutation.

### DETAILED DESCRIPTION

Disclosed herein are the results of comprehensive double alanine scanning of the entire protein sequence of capsid proteins from AAV8 and AAV9, and particular the biological properties of all resulting mutants, including tropism in mice, in vitro and in vivo transduction efficiency, and pharmacokinetic profiles following intravenous injection into mice. The biological properties were determined using AAV Barcode-Seq technology, described in Adachi et al, Nat Commun 5, 3075 (2014); incorporated by reference herein and include brain, heart, and muscle targeting and liver detargeting phenotypes, receptor binding, pharmacokinetics, and viral genome processing in the cell.

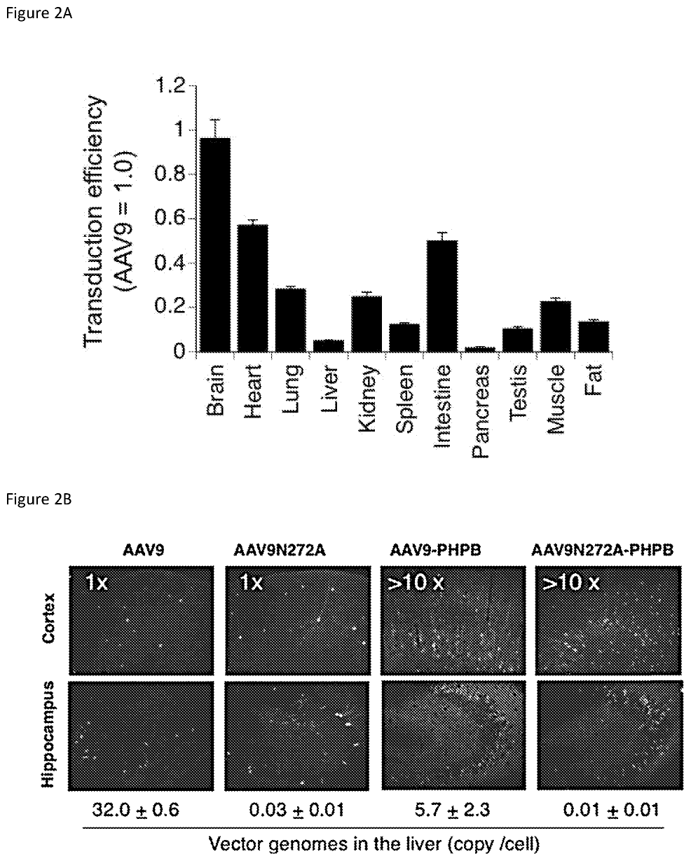

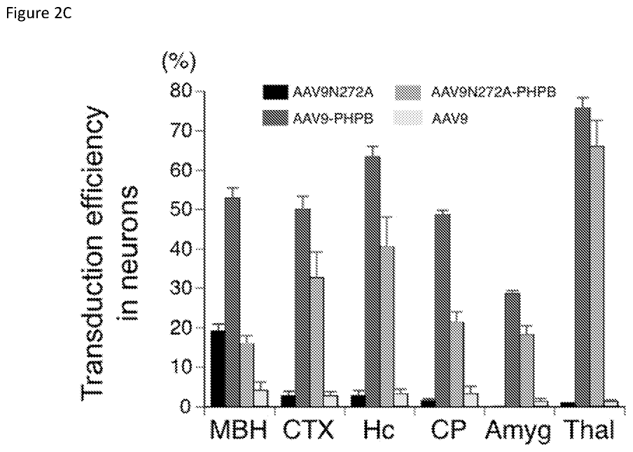

Included in this disclosure is a brain-targeting/liver-detargeting AAV9 vector that can transduce to the brain more than 10 times better than AAV9 while detargeting the liver following intravenous injection. This vector is termed AAV9-N272A-PHPB and includes an N272A mutation in the AAV9-PHPB capsid (described in Deverman et al, Nat Biotechnol 34, 204-209 (2016); which is incorporated by reference herein). An N272A mutation was introduced into AAV9-PHPB which transduces the brain very efficiently following intravenous injection of the vector. AAV9N272A-PHPB can transduce the brain more than 10 times better than the wild-type AAV9. Introduction of the N272 mutation resulted in >1000-fold less liver transduction compared to the wild-type AAV9 (FIG. 2).

AAV9N272A-PHPB represents an example of this type of knowledge-based approach, and similar approaches to create novel AAV capsids with combined phenotypes will be possible based on the information disclosed herein.

AAV vectors have widely been used in preclinical animal studies and have been used in clinical trials as promising gene delivery vehicles. Robust AAV serotypes such as AAV8 and AAV9 vectors can efficiently transduce various non-hepatic tissues such as the central nervous system, the heart and the muscle following intravenous injection of a high dose of vector and therefore has opened a new avenue for the treatment of various diseases involving the brain, heart and muscle that currently do not have effective therapies. However, the tropism of the currently available vectors is promiscuous even though the vectors can transduce the brain, heart, muscle and other organs efficiently following intravenous injection of the robust serotype vectors and their derived mutants. The problem of particular importance is that the vectors can inevitably be disseminated to the liver at high levels, which potentially causes various clinical problems including cytotoxic T lymphocyte (CTL)-induced hepatotoxicity and an increased risk of liver carcinogenesis. Therefore, liver-detargeting is very important for gene therapy for non-hepatic diseases. Disclosed herein are AAV capsid mutant vectors that can effectively transduce the target non-hepatic organs while detargeting the liver.

Disclosed herein is double alanine scanning of the entire regions of the AAV8 and AAV9 capsids using the AAV Barcode-Seq technology, and collected a comprehensive set of biological phenotypes of all the mutants.

Disclosed herein are liver-detargeting mutants that retain the ability to transduce the brain, heart, muscle and/or other non-hepatic organs at substantial levels comparable to the parental robust AAV serotypes. These include: AAV8G28A, AAV8P30A/K31A, AAV8P32A/P33A, AAV8T265A/S266A, AAV8T270A, AAV8N271A/D272A, AAV8N273A/T274A, AAV8Y275A/F276A, AAV8G277A/Y278A, AAV8S387A/Q388A, AAV8W505A, AAV8T506A, AAV8T528A/H529A, and AAV8K530A/D531A; and AAV9W22A/W23A, AAV9K258A/Q259A, AAV9S268A/S269S, and AAV9N272A. AAV8G28A and AAV8P30A/K31A efficiently transduce skeletal muscle while detargeting the liver. AAV9W22A and AAV9W22A/W23A all exclusively transduce the heart while detargeting the liver and other organs. AAV9N272A transduces to the brain comparably to the wild-type AAV9 while detargeting the liver.

Disclosed herein are AAV8 and AAV9 mutants that detarget all organs. These include AAV8W10A/L11A, AAV8G44A/L45A, AAV8V46A/L47A, AAV8P48A/G49A, AAV8Y50A/K51A, AAV8Y52A/L53A, AAV8G54A/P55A, AAV8F56A/N57A, AAV8G58A/L59A, AAV8D60A/K61A, AAV8D69A, AAV8E74A/H75A, AAV8D76A/K77A, AAV8Y79A, AAV8N88A/P89A, AAV8Y90A/L91A, AAV8R92A/Y93A, AAV8D97A, AAV8F100A/Q101A, AAV8V118A/F119A, AAV8K122A/K123A, AAV8K142A/K143A, AAV8R144A/P145A, AAV8R169A/K170A, AAV8R171A/L172A, AAV8N255A/H256A, AAV8K259A/Q260A, AAV8G267A/G268A, AAV81335A, AAV8L339A/T340A, AAV8L383A/N384A, AAV8N385A/G386A, AAV8G391A/R392A, AAV8D532A/E533A, AAV8S564A/E565A, AAV8E566A/E567A, AAV8T704A/S705A, AAV8N706A/Y707A, AAV8Y708A/K709A, AAV8S712A/V713A, AAV8V717A, AAV8P726A/R727A, AAV9W10A/L11A, AAV9G44A/L45A, AAV9V46A/L47A, AAV9P48A/G49A, AAV9Y50A/K51A, AAV9Y52A/L53A, AAV9G54A/P55A, AAV9G56A/N57A, AAV9G58A/L59A, AAV9G62A/E63A, AAV9D69A, AAV9E74A/H75A, AAV9D76A/K77A, AAV9Y79A, AAV9Y90A/L91A, AAV9K92A/Y93A, AAV9D97A, AAV9F100A/Q101A, AAV9V118A/F119A, AAV9K122A/K123A, AAV9K142A/K143A, AAV9R144A/P145A, AAV9K168A/K169A, AAV9R170A/L171A, AAV9G222A/S223A, AAV9N254A/H255A, AAV9N262A/S263A, AAV9T264A/S265A, AAV9G266A/G267A, AAV9N270A/D271A, AAV91334A, and AAV9L338A/T339A.

Disclosed herein are AAV mutants that can transduce the liver but detarget other organs. These include AAV8G86A/D87A and AAV8N94A/H95A. The AAV9 versions of these mutations (AAV9G84A/D85A and AAV9N94A/H95A) also transduce the liver but not as robustly as the AAV8 mutants.

Disclosed herein are AAV9N270A/D271A and AAV9N272A mutants which bind to the surface of CHO Pro5 cells but do not bind to the surface of CHO Lec2 cells indicating that these residues are responsible for binding to galactose. AAV9N270A/D271A detargets all organs. AAV9N272A allows efficient transduction to the brain while detargeting the liver.

Disclosed herein are a total of 35 AAV9 capsid mutants that bind to CHO Lec2 cells but transduce the cells at levels that are only less than 10% of the level of the wild-type AAV9. These mutants bind to terminal galactose in the cell surface glycan chains, but this binding does not result in the expression of viral genomes. These mutants are classified into two types (Type I and Type II) based on the quantity of viral genome DNA and viral genome transcripts recovered from the cells 2 days post-infection.

Type I AAV9 galactose binding mutants bind to cell surface galactose and can be internalized in the cell. However, the viral genomes become degraded resulting in the failure of viral genome expression. Such mutants include AAV9K258A/Q259A, AAV9N262A/S263A, AAV9T264A/S265A, AAV9G266A/G267A, and AAV9S268A/S269A.

Type II AAV9 galactose binding mutants bind to cell surface galactose and can be internalized in the cell. However, the majority of the viral genomes that enter the cell stay transcriptionally inactive by an unknown mechanism. Such mutants include AAV9W10A/L11A, AAV9G44A/L45A, AAV9V46A/L47A, AAV9P48A/G49A, AAV9Y50A/K51A, AAV9Y52A/L53A, AAV9G54A/P55A, AAV9G56A/N57A, AAV9G58A/L59A, AAV9D60A/K61A, AAV9G62A/E63A, AAV9N66A, AAV9D69A, AAV9E74A/H75A, AAV9D76A/K77A, AAV9Y79A, AAV9Q82A/L83A, AAV9G86A/D87A, AAV9N88A/P89A, AAV9Y90A/L91A, AAV9K92A/Y93A, AAV9N94A/H95A, AAV9D97A, AAV9F100A/Q101A, AAV9L104A/K105A, AAV9V118A/F119A, AAV9K122A/K123A, AAV9G222A/S223A, and AAV91334A, AAV9L338A/T339A.

Disclosed herein are AAV capsid mutants that result in slower clearance of vector particles from blood circulation relative to the wildtype. Such mutants have 10 and 2 times higher concentrations in the blood 24 hours after intravenous injection than the wild-type AAV8 and AAV9, respectively. These mutants detarget the liver and promote accumulation of transcriptionally inactive vector genome DNA in the spleen. It should be noted that many of these mutants detarget organs globally but some of the mutants still retain the ability to transduce non-hepatic organs as described above. Such mutants include AAV8T265A/S266A, AAV8G267A/G268A, AAV8T270A, AAV8N271A/D272A, AAV8N273A/T274A, AAV8L383A/N384A, AAV8N385A/G386A, AAV8S387A/Q388A, AAV8W505A, AAV8T506A, AAV8T528A/H529A, AAV8Y708A/K709A, AAV8P726A/R727A, AAV9G222A/S223A, AAV9N254A/H255A, AAV9K258A/Q259A, AAV9N262A/S263A, AAV9T264A/S265A, AAV9S268A/S269A, AAV9N270A/D271A, AAV9N272A, and AAV91334A.

The term “AAV vector” as used herein means any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. The term “AAV vector” may be used to refer to an AAV type viral particle (or virion) comprising at least a nucleic acid molecule encoding a protein of interest.

Additionally, the AAVs disclosed herein may be derived from various serotypes, including combinations of serotypes (e.g., “pseudotyped” AAV) or from various genomes (e.g., single-stranded or self-complementary). In particular embodiments, the AAV vectors disclosed herein may comprise desired proteins or protein variants. A “mutant” as used herein, refers to an amino acid sequence that is altered by one or more amino acids relative to one of the amino acid sequences disclosed in the Sequence Listing provided herein (each alteration can be referred to as a “mutation”). The mutant may have “conservative” mutations, wherein a substituted amino acid has similar structural or chemical properties. Such conservative mutations include the substitutions in the table below.

| Original Amino Acid | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Analogous minor variations may also include amino acid deletions or insertions, or both. Any of the variants described herein—even those comprising specified mutations—can be mutated by one or more amino acids relative to the protein described herein, provided that the mutated protein has the same or a similar phenotype to the protein described herein (e.g. as the result of a conservative mutation). One of skill in the art in light of this disclosure can create and test such mutated proteins for phenotype. Polynucleotides encoding the proteins of the present disclosure are not explicitly disclosed herein but can be readily inferred by one of skill in the art in light of this disclosure. The polynucleotides of the present disclosure can be composed of either RNA or DNA. The disclosure also encompasses those polynucleotides that are complementary in sequence to the polynucleotides disclosed herein.

Because of the degeneracy of the genetic code, a variety of different polynucleotide sequences can encode the proteins of the present disclosure. In addition, it is well within the skill of a person trained in the art to create alternative polynucleotide sequences encoding the same, or essentially the same, proteins disclosed herein. These alternative polynucleotide sequences are within the scope of the current disclosure. As used herein, references to “essentially the same” sequence refers to sequences which encode amino acid substitutions, deletions, additions, or insertions which do not eliminate or substantially alter the detectability or the activity of the polypeptide encoded by the polynucleotides of the present disclosure.

Polynucleotide and polypeptide sequences of the current disclosure can also be defined in terms of particular identity and/or similarity with certain polynucleotides and polypeptides described herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical as compared to a sequence disclosed herein. Unless otherwise specified, as used herein percent sequence identity and/or similarity of two sequence scan be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used.

Methods of producing AAV vectors as disclosed herein are well known in the art, including methods, for example, using packaging cells, auxiliary viruses or plasmids, and/or baculovirus systems (see, e.g., Samulski et al., J. Virology 63, 3822 (1989); Xiao et al., J. Virology 72, 2224 (1998); Inoue et al., J. Virol. 72, 7024 (1998); WO1998/022607; and WO2005/072364; all of which are incorporated by reference herein).

Methods of producing pseudotyped AAV vectors are also known (see, e.g., WO00/28004; incorporated by reference herein), as well as various modifications or formulations of AAV vectors, to reduce their immunogenicity upon in vivo administration (see, e.g., WO01/23001; WO00/73316; WO04/112727; WO05/005610; and WO99/06562; all of which are incorporated by reference herein). In some embodiments, AAV vectors can be prepared or derived from various serotypes of AAVs which may be mixed together or mixed with other types of viruses to produce chimeric (e.g., pseudotyped) AAV viruses.

In particular embodiments, the AAV vector can be a human serotype AAV vector. In such embodiments, a human AAV can be derived from any known serotype, e.g., from any one of serotypes 1-11, for instance from AAV1, AAV2, AAV4, AAV6, or AAV9. One example of such an AAV vector includes a vector comprising a nucleic acid molecule comprising an ITR and packaging sequence, operatively linked to a nucleic acid encoding an expression cassette for a protein of interest, and a nucleic acid encoding a protein of interest in an AAV9-de rived capsid that differs from SEQ ID NO: 1 or SEQ ID NO: 2 herein by one or more amino acids, including SEQ ID NOs: 3-109 as provided herein.

The AAV vectors disclosed herein can include a nucleic acid encoding a protein of interest. In various embodiments, the nucleic acid also may include one or more regulatory sequences allowing expression and, in some embodiments, secretion of the protein of interest, such as e.g., a promoter, enhancer, polyadenylation signal, an internal ribosome entry site (IRES), a sequence encoding a protein transduction domain (PTD), and the like. Thus, in some embodiments, the nucleic acid may comprise a promoter region operably linked to the coding sequence to cause or improve expression of the protein of interest in infected cells. Such a promoter may be ubiquitous, cell- or tissue-specific, strong, weak, regulated, chimeric, etc., for example to allow efficient and stable production of the protein in the infected tissue. The promoter may be homologous to the encoded protein, or heterologous, although generally promoters of use in the disclosed methods are functional in human cells. Examples of regulated promoters include, without limitation, Teton/off element-containing promoters, rapamycin inducible promoters, tamoxifen-inducible promoters, and metallothionein promoters. Other promoters that may be used include promoters that are tissue specific for tissues such as kidney, spleen, and pancreas. Examples of ubiquitous promoters include viral promoters, particularly the CMV promoter, the RSV promoter, the SV40 promoter, etc., and cellular promoters such as the PGK (phosphoglycerate kinase) promoter and the 3-actin promoter.

In some embodiments of the AAV vectors disclosed herein, one or more feedback elements may be used to dampen over-expression of the protein of interest. For example, some embodiments of the AAV vectors may include one or more siRNA sequences that would target the exogenous transcript. In other embodiments, the AAV vector may include one or more additional promoters that may be recognized by inhibitory transcription factors. In various embodiments, the AAV vectors disclosed herein may comprise a construct that may create a homoeostatic feedback loop that may maintain expression levels of the protein of interest at a physiological level.

In various embodiments, the AAV vectors disclosed herein can comprise a nucleic acid that may include a leader sequence allowing secretion of the encoded protein. In some embodiments, fusion of the transgene of interest with a sequence encoding a secretion signal peptide (usually located at the N-terminal of secreted polypeptides) may allow the production of the therapeutic protein in a form that can be secreted from the transduced cell. Examples of such signal peptides include the albumin, the β-glucuronidase, the alkaline protease or the fibronectin secretory signal peptides.

As described herein, effective and long term expression of the rapeutic proteins of interest in brain, heart, lung, skeletal muscle, kidney, spleen, or pancreas can be achieved with non-invasive techniques, through peripheral administration of certain AAV vectors, such as a non-AAV9 vector with AAV9 sequences. Such peripheral administration may include any administration route that does not necessitate direct injection into brain, heart, lung, skeletal muscle, kidney, spleen, or pancreas. More particularly, peripheral administration may include systemic injections, such as intramuscular, intravascular (such as intravenous) intraperitoneal, intra-arterial, or subcutaneous injections. In some embodiments, peripheral administration also may include oral administration (see, for instance, WO96/40954; incorporated by reference herein), delivery using implants, (see, for instance, WO01/91803; incorporated by reference herein), or administration by instillation through the respiratory system, e.g., using sprays, aerosols or any other appropriate formulations.

In various embodiments, the desired doses of the AAV vectors may be easily adapted by the skilled artisan, e.g., depending on the disease condition, the subject, the treatment schedule, etc. In some embodiments, from 10⁵ to 10¹² viral genomes are administered per dose, for example, from 10⁶ to 10¹¹, from 10⁷ to 10¹¹, or from 10⁸ to 10¹¹. In other embodiments, exemplary doses for achieving the rapeutic effects may include virus titers of at least about 10⁵, 10⁶, 10⁷, 10⁸, 10⁹, 10¹⁰ or 10¹¹ viral genomes or more. Virus titer may also be expressed in terms of transducing units, which may be readily calculated by those of skill in the art.

In various embodiments, the AAV vectors disclosed herein may be administered in any suitable form, for instance, either as a liquid solution or suspension, as a solid form suitable for solution or suspension in liquid prior to injection, as a gel or as an emulsion. The vectors may be formulated with any appropriate and pharmaceutically acceptable excipient, carrier, adjuvant, diluent, etc. For instance, for injection, a suitable carrier or diluent may be an isotonic solution, a buffer, sterile and pyrogen-free water, or, for instance, a sterile and pyrogen-free phosphate buffered saline solution. For inhalation, the carrier may be in particulate form.

The vectors may be administered in a “therapeutically-effective” amount, e.g., an amount that is sufficient to alleviate (e.g., decrease, reduce) at least one of the symptoms associated with a disease state, or to provide improvement in the condition of the subject. In some embodiments, repeated administrations may be performed, for instance using the same or a different peripheral administration route and/or the same vector or a distinct vector.

To better understand AAV capsid biology, a novel comprehensive high-throughput reverse genetics method was established that integrates DNA barcoding and next generation sequencing technologies. This approach has been termed “AAV Barcode-Seq”. DNA-barcoded AAV capsid libraries with defined capsid mutations are generated and amino acid sequence.

AAV capsid phenotype relationship data is collected in vitro and in vivo. A number of amino acids that play important roles in capsid assembly, receptor binding, tropism, blood clearance rates and antibody recognition have been identified (Adachi K et al, Nat Commun 5, 3075 (2014); WO 2013-159036; and WO 2013-170078, all of which are incorporated by reference herein). Disclosed herein is a more advanced approach that uses DNA/RNA-barcoded AAV libraries. In this new approach, DNA barcodes are expressed as RNA barcodes under the control of the human U6 snRNA RNA polymerase Ill promoter. By analyzing the quantity of expressed RNA barcodes by reverse transcription (RT)-PCR, viral gene expression can be quantified. Such expression is important for assessment of AAV vector transduction (i.e., gene delivery resulting in vector genome expression) because viral genome DNA and viral genome transcripts can be discordant.

In earlier work, pharmacokinetic profiles as well as in vivo transduction efficiency of various AAV serotypes and capsid-modified variants and mutants administered intravenously in mice were established (Kotchey N M et al, Mol Ther 19, 1079-1089 (2011); incorporated by reference herein). Rapid clearance of exogenous agents from the bloodstream primarily by Kupffer cells in the liver has been a major hurdle in systemic treatments with such agents. This is particularly the case with adenoviral vectors and cationic non-viral vectors (Alemany R et al, J Gen Virol 81, 2605-2609; 2000; Fenske D B et al, Curr Opin Mol Ther 3, 153-158 (2001); Li S D and Huang L, Gene Ther 13, 1313-1319 (2006); Manickan E et al, Molt her 13, 108-117; 2006; Schagen F H et al, Hum Gene Ther 19, 783-794 (2008); Shayakhmetov D M et al, J Virol 78, 5368-5381 (2004); Tao N et al, Mol Ther 3, 28-35 (2001); Xu Z L et al, Adv Drug Deliv Rev 57, 781-802 (2005); all of which are incorporated by reference herein.

### EXAMPLES

The following examples are for illustration only. In light of this disclosure, those of skill in the art will recognize that variations of these examples and other examples of the disclosed invention be possible without undue experimentation.

### Example 1—AAV Barcode-Seq Data Showing the Biological Phenotypes of AAV8 Alanine Mutants

Two DNA/RNA-barcoded AAV libraries containing AAV8 double alanine mutants and reference controls AAV9, AAV8 and AAV2R585E, (AAV Stock Identification Numbers (IDs) 538 and 539) were injected into 8-week-old C57BL/6 male mice. For the tissue transduction analysis (n=3 per library), the mice were injected with each AAV library at a dose of 2×10¹³ vector genome (vg) per mouse. Total DNA and total RNA were extracted from eleven major tissues collected 6 weeks post-injection, and subjected to the DNA Barcode-Seq and RNA Barcode-Seq analyses. The vector genomes packaged in this library express viral clone-specific 12 nucleotide-long RNA barcodes under the control of the human U6 small nuclear RNA gene promoter. This allows us to analyze quantitatively vector genome DNA copy numbers and vector genome RNA transcripts in samples by Illumina barcode sequencing. For the pharmacokinetic analysis (n=2 per library), the mice were injected with each AAV library at a dose of 1×10¹³ vg per mouse. Blood samples were collected 1 min, 10 min, 30 min, 1 h, 4 h, 8 h, 24 h and 72 h post injection, and subjected to the DNA Barcode-Seq analysis to quantify viral genome copy numbers in the blood samples. These two libraries coverall the capsid outer surface-exposed amino acids. The mutants that do not produce viable virions were excluded from the analysis. The values in the table represent the averages from replicated experiments. All the values are normalized with the values obtained from AAV8, one of the reference controls.

A DNA/RNA-barcoded AAV library containing AAV9 double alanine mutants and reference controls AAV9 and AAV2R585E, (AAV Stock ID 507) was injected into 8-week-old C57BL/6 male mice. For the tissue transduction analysis (n=3), the mice were injected with the AAV library at a dose of 2×10¹³ vg per mouse. Total DNA and total RNA were extracted from eleven major tissues collected 6 weeks post-injection and subjected to the DNA Barcode-Seq and RNA Barcode-Seq analyses. For the pharmacokinetic analysis (n=2), the mice were injected with the AAV library at a dose of 1×10¹³ vg per mouse. Blood samples were collected 1 min, 10 min, 30 min, 1 h, 4 h, 8 h, 24 h and 72 h post injection, and subjected to the DNA Barcode-Seq analysis to quantify viral genome copy numbers in the blood samples. For the in vitro cell surface binding and transduction assays, each library was applied on the cells of interest at a multiplicity of infection (MOI) of 10⁴ and subjected to the procedure described in Adachi et al 2014 supra.

The library covers the N-terminal half of the AAV9 capsid. The DNA Barcode-Seq data for the C-terminal half of the AAV9 capsid was published in Adachi et al 2014 supra. The mutants that do not produce viable virions were excluded from the analysis. The values in the table represent the averages from replicated experiments. All the values are normalized with the values obtained from AAV9, one of the reference controls.

Table 1 shows AAV8 capsid mutants with liver transduction efficiencies less than 10% of the wild-type AAV8 identified via RNA Barcode-Seq (units in vector genome expression per tissue relative to the wild-type AAV8). Liver detargeting mutants that can also transduce non-hepatic organs to are shaded. Abbreviations: Br=brain, H=heart, Lu=lung, Lv=liver, K=kidney, S=spleen, I=intestine, T=testis, M=skeletal muscle, F=fat

| Mutation | Br | H | Lu | Lv | K | S | I | T | M | F |
|---|---|---|---|---|---|---|---|---|---|---|
| AAV9 wt | 3.66 | 1.25 | 1.19 | 0.28 | 1.21 | 1.14 | 2.00 | 1.09 | 0.37 | 1.14 |
| R585E | 1.89 | 0.63 | 0.21 | 0.01 | 0.12 | 0.04 | 0.32 | 0.06 | 0.02 | 0.05 |
| AAV8 wt | 1.00 | 1.00 | 1.00 | 1.00 | 1.00 | 1.00 | 1.00 | 1.00 | 1.00 | 1.00 |
| W10A/L11A | 0.00 | 0.12 | 0.00 | 0.00 | 0.00 | 0.00 | 0.00 | 0.00 | 0.00 | 0.01 |
| P48A/G49A | 0.00 | 0.05 | 0.00 | 0.00 | 0.00 | 0.00 | 0.00 | 0.00 | 0.00 | 0.00 |
| Y50A/K51A | 0.00 | 0.03 | 0.00 | 0.00 | 0.01 | 0.00 | 0.00 | 0.00 | 0.00 | 0.01 |
| Y52A/L53A | 0.00 | 0.01 | 0.00 | 0.00 | 0.00 | 0.00 | 0.00 | 0.00 | 0.00 | 0.00 |
| G54A/P55A | 0.00 | 0.02 | 0.00 | 0.00 | 0.00 | 0.00 | 0.00 | 0.00 | 0.00 | 0.00 |
| F56A/N57A | 0.00 | 0.04 | 0.00 | 0.00 | 0.00 | 0.00 | 0.00 | 0.00 | 0.00 | 0.00 |
| G58A/L59A | 0.00 | 0.03 | 0.00 | 0.00 | 0.00 | 0.00 | 0.00 | 0.00 | 0.00 | 0.03 |
| D69A | 0.00 | 0.01 | 0.00 | 0.00 | 0.00 | 0.00 | 0.00 | 0.00 | 0.00 | 0.00 |
| E74A/H75A | 0.00 | 0.04 | 0.00 | 0.00 | 0.00 | 0.00 | 0.00 | 0.00 | 0.00 | 0.00 |
| D76A/K77A | 0.00 | 0.02 | 0.00 | 0.00 | 0.00 | 0.00 | 0.00 | 0.00 | 0.00 | 0.01 |
| Y79A | 0.00 | 0.01 | 0.01 | 0.00 | 0.00 | 0.00 | 0.00 | 0.00 | 0.00 | 0.01 |
| Y90A/L91A | 0.00 | 0.01 | 0.00 | 0.00 | 0.00 | 0.00 | 0.00 | 0.00 | 0.00 | 0.00 |
| D97A | 0.00 | 0.02 | 0.00 | 0.00 | 0.00 | 0.00 | 0.00 | 0.00 | 0.00 | 0.00 |
| F100A/Q101A | 0.00 | 0.01 | 0.00 | 0.00 | 0.00 | 0.00 | 0.00 | 0.00 | 0.00 | 0.00 |
| V118A/F119A | 0.00 | 0.03 | 0.00 | 0.00 | 0.01 | 0.00 | 0.00 | 0.00 | 0.00 | 0.01 |
| K122A/K123A | 0.00 | 0.02 | 0.00 | 0.00 | 0.00 | 0.00 | 0.00 | 0.00 | 0.00 | 0.00 |
| K142A/K143A | 0.00 | 0.01 | 0.00 | 0.00 | 0.00 | 0.00 | 0.00 | 0.00 | 0.00 | 0.00 |
| R144A/P145A | 0.00 | 0.00 | 0.00 | 0.00 | 0.00 | 0.00 | 0.00 | 0.00 | 0.00 | 0.00 |
| R169A/K170A | 0.00 | 0.04 | 0.01 | 0.00 | 0.01 | 0.01 | 0.01 | 0.01 | 0.00 | 0.01 |
| R171A/L172A | 0.00 | 0.02 | 0.01 | 0.00 | 0.01 | 0.01 | 0.01 | 0.01 | 0.00 | 0.01 |
| T265A/S266A | 0.49 | 0.58 | 0.19 | 0.00 | 0.17 | 0.03 | 0.45 | 0.06 | 0.06 | 0.32 |
| G267A/G268A | 0.00 | 0.11 | 0.02 | 0.00 | 0.02 | 0.00 | 0.01 | 0.01 | 0.00 | 0.02 |
| N273A/T274A | 0.07 | 0.20 | 0.12 | 0.00 | 0.11 | 0.02 | 0.23 | 0.03 | 0.04 | 0.09 |
| Y275A/F276A | 0.06 | 0.31 | 0.10 | 0.00 | 0.06 | 0.08 | 0.08 | 0.06 | 0.02 | 0.07 |
| G277A/Y278A | 0.00 | 0.33 | 0.18 | 0.00 | 0.21 | 0.25 | 0.31 | 0.25 | 0.02 | 0.30 |
| L383A/N384A | 0.00 | 0.02 | 0.00 | 0.00 | 0.02 | 0.00 | 0.12 | 0.00 | 0.00 | 0.02 |
| N385A/G386A | 0.12 | 0.15 | 0.06 | 0.00 | 0.04 | 0.01 | 0.15 | 0.02 | 0.03 | 0.09 |
| G391A/R392A | 0.00 | 0.00 | 0.00 | 0.00 | 0.00 | 0.00 | 0.00 | 0.00 | 0.00 | 0.03 |
| T704A/S705A | 0.02 | 0.01 | 0.00 | 0.00 | 0.04 | 0.00 | 0.00 | 0.00 | 0.00 | 0.02 |
| N706A/Y707A | 0.00 | 0.00 | 0.00 | 0.00 | 0.00 | 0.00 | 0.01 | 0.00 | 0.00 | 0.00 |
| Y708A/K709A | 0.00 | 0.28 | 0.07 | 0.00 | 0.05 | 0.03 | 0.14 | 0.02 | 0.02 | 0.06 |
| P726A/R727A | 0.20 | 0.02 | 0.01 | 0.00 | 0.02 | 0.00 | 0.01 | 0.00 | 0.01 | 0.04 |
| W22A/W23A | 0.00 | 0.25 | 0.00 | 0.01 | 0.03 | 0.03 | 0.00 | 0.00 | 0.01 | 0.07 |
| G44A/L45A | 0.00 | 0.03 | 0.00 | 0.01 | 0.00 | 0.00 | 0.00 | 0.00 | 0.00 | 0.00 |
| V46A/L47A | 0.00 | 0.01 | 0.00 | 0.01 | 0.00 | 0.00 | 0.00 | 0.00 | 0.00 | 0.00 |
| R92A/Y93A | 0.00 | 0.01 | 0.00 | 0.01 | 0.01 | 0.00 | 0.00 | 0.00 | 0.00 | 0.01 |
| T270A | 0.18 | 0.40 | 0.13 | 0.01 | 0.24 | 0.05 | 0.56 | 0.05 | 0.02 | 0.19 |
| L339A/T340A | 0.00 | 0.00 | 0.00 | 0.01 | 0.01 | 0.00 | 0.01 | 0.00 | 0.00 | 0.01 |
| W505A | 0.41 | 0.57 | 0.15 | 0.01 | 0.19 | 0.05 | 0.46 | 0.09 | 0.11 | 0.22 |
| D532A/E533A | 0.01 | 0.15 | 0.06 | 0.01 | 0.16 | 0.01 | 0.49 | 0.05 | 0.09 | 0.14 |
| S564A/E565A | 0.01 | 0.00 | 0.01 | 0.01 | 0.01 | 0.01 | 0.06 | 0.00 | 0.00 | 0.01 |
| N271A/D272A | 0.75 | 0.86 | 0.19 | 0.02 | 0.24 | 0.07 | 0.48 | 0.16 | 0.04 | 0.25 |
| S387A/Q388A | 0.26 | 0.86 | 0.19 | 0.02 | 0.33 | 0.10 | 0.43 | 0.07 | 0.15 | 0.33 |
| T528A/H529A | 0.28 | 0.84 | 0.17 | 0.03 | 0.36 | 0.30 | 0.20 | 0.06 | 0.16 | 0.29 |
| E566A/E567A | 0.01 | 0.01 | 0.01 | 0.03 | 0.01 | 0.01 | 0.01 | 0.01 | 0.00 | 0.01 |
| I335A | 0.01 | 0.04 | 0.03 | 0.04 | 0.02 | 0.01 | 0.05 | 0.05 | 0.02 | 0.04 |
| D60A/K61A | 0.00 | 0.05 | 0.01 | 0.05 | 0.02 | 0.02 | 0.02 | 0.04 | 0.03 | 0.19 |
| N255A/H256A | 0.01 | 0.11 | 0.02 | 0.05 | 0.04 | 0.00 | 0.05 | 0.02 | 0.02 | 0.05 |
| V717A | 0.02 | 0.06 | 0.02 | 0.05 | 0.06 | 0.04 | 0.10 | 0.05 | 0.10 | 0.09 |
| K259A/Q260A | 0.00 | 0.21 | 0.06 | 0.06 | 0.02 | 0.04 | 0.18 | 0.06 | 0.03 | 0.11 |
| K530A/D531A | 1.29 | 0.96 | 0.28 | 0.07 | 0.40 | 0.21 | 1.12 | 0.41 | 0.19 | 0.54 |
| S712A/V713A | 0.00 | 0.00 | 0.04 | 0.07 | 0.10 | 0.04 | 0.01 | 0.07 | 0.05 | 0.13 |
| P32A/K33A | 0.26 | 0.50 | 0.40 | 0.08 | 0.36 | 0.47 | 0.40 | 0.22 | 0.44 | 0.50 |
| N88A/P89A | 0.06 | 0.06 | 0.01 | 0.09 | 0.02 | 0.01 | 0.05 | 0.01 | 0.01 | 0.19 |
| T506A | 1.52 | 1.57 | 0.63 | 0.10 | 0.75 | 0.40 | 2.03 | 0.42 | 0.36 | 0.87 |
| P30A/K31A | 0.83 | 0.94 | 0.74 | 0.13 | 0.70 | 0.74 | 0.61 | 0.46 | 0.76 | 0.80 |
| G28A | 0.44 | 0.81 | 0.47 | 0.14 | 0.56 | 0.63 | 0.72 | 0.61 | 0.91 | 0.79 |

Table 2 shows Liver detargeting AAV9 alanine mutants. AAV9 mutants with liver transduction values less than 10% of the wild-type AAV9 values are shown included. Liver detargeting mutants that can also transduce non-hepatic organs to are shaded. Abbreviations: same as for Table 1 except Pa=pancreas.

| Mutation | Br | H | Lu | Lv | K | S | I | Pa | T | M | F |
|---|---|---|---|---|---|---|---|---|---|---|---|
| wild-type | 1.00 | 1.00 | 1.00 | 1.00 | 1.00 | 1.00 | 1.00 | 1.00 | 1.00 | 1.00 | 1.00 |
| R585E | 0.41 | 0.35 | 0.16 | 0.01 | 0.14 | 0.04 | 0.13 | 0.00 | 0.03 | 0.12 | 0.03 |
| K142A/K143A | 0.00 | 0.00 | 0.00 | 0.00 | 0.00 | 0.00 | 0.00 | 0.00 | 0.00 | 0.00 | 0.00 |
| R144A/P145A | 0.00 | 0.00 | 0.00 | 0.00 | 0.00 | 0.00 | 0.00 | 0.00 | 0.00 | 0.00 | 0.00 |
| K122A/K123A | 0.00 | 0.00 | 0.00 | 0.00 | 0.01 | 0.00 | 0.00 | 0.00 | 0.00 | 0.00 | 0.00 |
| K168A/K169A | 0.00 | 0.00 | 0.00 | 0.00 | 0.00 | 0.00 | 0.00 | 0.00 | 0.00 | 0.00 | 0.00 |
| W10A/L11A | 0.00 | 0.07 | 0.00 | 0.00 | 0.00 | 0.01 | 0.00 | 0.00 | 0.00 | 0.03 | 0.01 |
| L338A/T339A | 0.00 | 0.00 | 0.00 | 0.00 | 0.00 | 0.00 | 0.00 | 0.00 | 0.01 | 0.00 | 0.00 |
| P48A/G49A | 0.00 | 0.00 | 0.00 | 0.00 | 0.00 | 0.00 | 0.00 | 0.00 | 0.00 | 0.00 | 0.00 |
| D69A | 0.00 | 0.00 | 0.00 | 0.00 | 0.00 | 0.00 | 0.00 | 0.00 | 0.00 | 0.00 | 0.00 |
| D76A/K77A | 0.00 | 0.00 | 0.00 | 0.00 | 0.00 | 0.00 | 0.00 | 0.00 | 0.00 | 0.00 | 0.00 |
| G222A/S223A | 0.00 | 0.00 | 0.00 | 0.00 | 0.00 | 0.00 | 0.00 | 0.00 | 0.00 | 0.00 | 0.00 |
| Y50A/K51A | 0.00 | 0.00 | 0.01 | 0.00 | 0.00 | 0.00 | 0.00 | 0.00 | 0.00 | 0.00 | 0.00 |
| G54A/P55A | 0.00 | 0.00 | 0.00 | 0.00 | 0.01 | 0.00 | 0.00 | 0.00 | 0.00 | 0.00 | 0.00 |
| G56A/N57A | 0.00 | 0.00 | 0.00 | 0.00 | 0.00 | 0.00 | 0.00 | 0.00 | 0.00 | 0.00 | 0.00 |
| E74A/H75A | 0.00 | 0.00 | 0.00 | 0.00 | 0.00 | 0.00 | 0.00 | 0.00 | 0.00 | 0.00 | 0.00 |
| Y79A | 0.00 | 0.00 | 0.00 | 0.00 | 0.00 | 0.00 | 0.01 | 0.00 | 0.00 | 0.00 | 0.00 |
| D97A | 0.00 | 0.00 | 0.00 | 0.00 | 0.00 | 0.00 | 0.00 | 0.00 | 0.00 | 0.00 | 0.00 |
| N262A/S263A | 0.10 | 0.19 | 0.07 | 0.00 | 0.06 | 0.01 | 0.08 | 0.00 | 0.03 | 0.09 | 0.01 |
| T264A/S265A | 0.04 | 0.10 | 0.02 | 0.00 | 0.03 | 0.01 | 0.01 | 0.01 | 0.01 | 0.07 | 0.01 |
| G266A/G267A | 0.01 | 0.01 | 0.01 | 0.00 | 0.00 | 0.01 | 0.01 | 0.01 | 0.00 | 0.00 | 0.01 |
| Y90A/L91A | 0.00 | 0.00 | 0.00 | 0.01 | 0.00 | 0.00 | 0.00 | 0.00 | 0.00 | 0.00 | 0.00 |
| V46A/L47A | 0.00 | 0.00 | 0.00 | 0.01 | 0.00 | 0.00 | 0.00 | 0.00 | 0.00 | 0.00 | 0.00 |
| Y52A/L53A | 0.00 | 0.00 | 0.01 | 0.01 | 0.00 | 0.00 | 0.00 | 0.00 | 0.00 | 0.00 | 0.00 |
| K92A/Y93A | 0.00 | 0.00 | 0.00 | 0.01 | 0.00 | 0.00 | 0.00 | 0.00 | 0.00 | 0.00 | 0.00 |
| F100A/Q101A | 0.00 | 0.00 | 0.00 | 0.01 | 0.00 | 0.00 | 0.00 | 0.00 | 0.00 | 0.00 | 0.00 |
| N270A/D271A | 0.01 | 0.05 | 0.01 | 0.01 | 0.01 | 0.01 | 0.01 | 0.01 | 0.01 | 0.02 | 0.01 |
| W22A/W23A | 0.03 | 0.51 | 0.03 | 0.01 | 0.02 | 0.09 | 0.02 | 0.07 | 0.01 | 0.07 | 0.05 |
| V118A/F119A | 0.00 | 0.02 | 0.00 | 0.01 | 0.00 | 0.00 | 0.00 | 0.00 | 0.00 | 0.01 | 0.01 |
| G44A/L45A | 0.00 | 0.00 | 0.00 | 0.01 | 0.00 | 0.00 | 0.00 | 0.00 | 0.00 | 0.00 | 0.00 |
| S268A/S269A | 0.22 | 0.26 | 0.12 | 0.01 | 0.07 | 0.02 | 0.13 | 0.01 | 0.03 | 0.15 | 0.06 |
| G58A/L59A | 0.00 | 0.01 | 0.01 | 0.01 | 0.00 | 0.00 | 0.00 | 0.00 | 0.00 | 0.01 | 0.01 |
| I334A | 0.01 | 0.03 | 0.02 | 0.02 | 0.02 | 0.02 | 0.02 | 0.00 | 0.02 | 0.01 | 0.03 |
| K258A/Q259A | 0.28 | 0.30 | 0.20 | 0.03 | 0.24 | 0.06 | 0.42 | 0.01 | 0.10 | 0.26 | 0.15 |
| N272A | 0.96 | 0.57 | 0.28 | 0.05 | 0.25 | 0.12 | 0.50 | 0.02 | 0.10 | 0.23 | 0.13 |
| G62A/E63A | 0.06 | 0.05 | 0.02 | 0.06 | 0.01 | 0.01 | 0.02 | 0.00 | 0.02 | 0.04 | 0.09 |
| R170A/L171A | 0.01 | 0.09 | 0.09 | 0.08 | 0.12 | 0.06 | 0.05 | 0.09 | 0.09 | 0.14 | 0.20 |
| N254A/H255A | 0.08 | 0.17 | 0.09 | 0.09 | 0.04 | 0.06 | 0.07 | 0.03 | 0.05 | 0.05 | 0.16 |

Table 3 shows mutants that are deficient in galactose binding. Indicated are AAV9 mutants that bind to Pro5 cells at a level comparable to the wild-type AAV9, but bind to Lec2 cells at less than 5% of the wild-type AAV9.

| | Cell binding | | | Transduction (DNA) | | | Transduction (RNA) | | |
|---|---|---|---|---|---|---|---|---|---|
| Mutation | Pro5 | Lec2 | 293 | Pro5 | Lec2 | 293 | Pro5 | Lec2 | 293 |
| wild-type | 1.00 | 1.00 | 1.00 | 1.00 | 1.00 | 1.00 | 1.00 | 1.00 | 1.00 |
| R585E | 1.37 | 0.04 | 0.56 | 0.43 | 0.10 | 0.80 | 0.02 | 0.01 | 0.76 |
| N272A | 0.98 | 0.03 | 0.88 | 0.11 | 0.04 | 0.29 | 0.01 | 0.01 | 0.19 |
| N270A/D271A | 0.96 | 0.03 | 1.60 | 0.17 | 0.06 | 0.29 | 0.00 | 0.00 | 0.00 |

Table 4 shows mutants that are deficient in post cell surface binding in CHO Lec2 cell transduction. AAV9 Type I galactose binding mutants as described herein are shaded. The other mutants (other than R585E) are AAV9 Type II galactose binding mutants as described herein.

| | Cell binding | | | Transduction (DNA) | | | Transduction (RNA) | | |
|---|---|---|---|---|---|---|---|---|---|
| Mutation | Pro5 | Lec2 | 293 | Pro5 | Lec2 | 293 | Pro5 | Lec2 | 293 |
| wild-type | 1.00 | 1.00 | 1.00 | 1.00 | 1.00 | 1.00 | 1.00 | 1.00 | 1.00 |
| R585E | 1.37 | 0.04 | 0.56 | 0.43 | 0.10 | 0.80 | 0.02 | 0.01 | 0.76 |
| T264A/S265A | 0.89 | 1.00 | 0.90 | 0.19 | 0.09 | 0.47 | 0.00 | 0.00 | 0.00 |
| G266A/G267A | 0.98 | 1.01 | 1.05 | 0.22 | 0.11 | 0.49 | 0.00 | 0.00 | 0.00 |
| D76A/K77A | 1.05 | 1.10 | 0.90 | 1.85 | 2.11 | 1.33 | 0.00 | 0.00 | 0.00 |
| V46A/L47A | 0.99 | 1.04 | 0.91 | 1.93 | 2.32 | 1.47 | 0.00 | 0.00 | 0.01 |
| G54A/P55A | 1.01 | 1.02 | 0.99 | 1.80 | 2.22 | 1.32 | 0.00 | 0.01 | 0.00 |
| L338A/T339A | 0.94 | 0.91 | 3.01 | 0.38 | 0.43 | 0.56 | 0.01 | 0.01 | 0.00 |
| N94A/H95A | 0.95 | 0.95 | 1.02 | 1.98 | 2.39 | 1.44 | 0.00 | 0.01 | 0.00 |
| K92A/Y93A | 1.03 | 1.02 | 1.04 | 1.92 | 2.35 | 1.48 | 0.00 | 0.01 | 0.01 |
| D69A | 1.01 | 1.05 | 1.08 | 1.82 | 2.18 | 1.33 | 0.00 | 0.01 | 0.00 |
| G222A/S223A | 1.24 | 1.05 | 1.87 | 0.51 | 0.60 | 0.90 | 0.01 | 0.01 | 0.01 |
| Y79A | 1.08 | 1.14 | 1.06 | 1.82 | 2.30 | 1.47 | 0.00 | 0.01 | 0.00 |
| D97A | 1.02 | 0.99 | 0.97 | 1.78 | 2.19 | 1.26 | 0.00 | 0.01 | 0.04 |
| S268A/S269A | 0.96 | 1.07 | 0.96 | 0.18 | 0.08 | 0.42 | 0.00 | 0.01 | 0.00 |
| Y50A/K51A | 1.05 | 1.10 | 1.10 | 1.91 | 2.27 | 1.42 | 0.01 | 0.01 | 0.00 |
| G56A/N57A | 0.84 | 0.85 | 0.89 | 1.89 | 2.29 | 1.29 | 0.00 | 0.01 | 0.00 |
| Y52A/L53A | 1.00 | 1.03 | 1.06 | 2.07 | 2.50 | 1.53 | 0.00 | 0.01 | 0.00 |
| Y90A/L91A | 1.01 | 1.08 | 1.08 | 1.94 | 2.16 | 1.38 | 0.01 | 0.01 | 0.00 |
| G58A/L59A | 1.03 | 1.16 | 1.01 | 1.85 | 2.09 | 1.34 | 0.01 | 0.01 | 0.00 |
| N262A/S263A | 0.93 | 0.93 | 0.88 | 0.14 | 0.06 | 0.40 | 0.00 | 0.01 | 0.07 |
| G62A/E63A | 0.97 | 0.98 | 1.03 | 1.53 | 1.79 | 1.04 | 0.01 | 0.01 | 0.06 |
| G86A/D87A | 1.02 | 1.10 | 0.94 | 1.82 | 2.18 | 1.26 | 0.01 | 0.01 | 0.00 |
| E74A/H75A | 0.96 | 0.97 | 0.97 | 1.80 | 2.10 | 1.32 | 0.01 | 0.01 | 0.01 |
| F100A/Q101A | 1.15 | 1.20 | 1.16 | 2.04 | 2.42 | 1.54 | 0.00 | 0.01 | 0.00 |
| G44A/L45A | 1.08 | 1.27 | 1.22 | 2.30 | 2.68 | 1.79 | 0.01 | 0.01 | 0.00 |
| P48A/G49A | 0.98 | 1.01 | 0.98 | 1.97 | 2.57 | 1.46 | 0.02 | 0.01 | 0.01 |
| V118A/F119A | 1.00 | 1.00 | 1.12 | 1.80 | 2.20 | 1.34 | 0.00 | 0.02 | 0.00 |
| K122A/K123A | 1.07 | 1.04 | 1.04 | 1.47 | 1.78 | 1.11 | 0.00 | 0.02 | 0.00 |
| N88A/P89A | 1.00 | 1.02 | 0.93 | 1.77 | 2.14 | 1.33 | 0.01 | 0.02 | 0.00 |
| N66A | 1.06 | 1.13 | 1.29 | 1.93 | 2.30 | 1.43 | 0.01 | 0.02 | 0.00 |
| Q82A/L83A | 1.07 | 1.16 | 1.10 | 1.84 | 2.13 | 1.39 | 0.02 | 0.04 | 0.09 |
| K258A/Q259A | 1.15 | 1.12 | 1.49 | 0.22 | 0.17 | 0.57 | 0.00 | 0.04 | 0.04 |
| D60A/K61A | 1.00 | 1.00 | 1.03 | 1.68 | 1.95 | 1.10 | 0.03 | 0.06 | 0.61 |
| I334A | 1.07 | 1.07 | 2.42 | 0.51 | 0.63 | 0.82 | 0.06 | 0.07 | 0.15 |
| L104A/K105A | 0.95 | 1.02 | 1.01 | 1.82 | 2.23 | 1.36 | 0.03 | 0.09 | 0.12 |
| W10A/L11A | 1.03 | 0.94 | 1.96 | 0.62 | 1.16 | 1.06 | 0.16 | 0.09 | 0.01 |

Table 5 shows AAV8 alanine mutations with slower blood clearance than the wild type. The listed mutations display retention in the blood at least 10 times higher than the wild-type AAV8 at 24 hours.

| Mutant | 1 m | 10 m | 30 m | 1 h | 4 h | 8 h | 24 h |
|---|---|---|---|---|---|---|---|
| AAV9 w/t | 0.93 | 0.94 | 1.07 | 1.48 | 2.20 | 3.70 | 5.28 |
| R585E | 0.91 | 0.94 | 1.23 | 1.52 | 3.22 | 8.22 | 16.97 |
| AAV8 w/t | 1.00 | 1.00 | 1.00 | 1.00 | 1.00 | 1.00 | 1.00 |
| T265A/S266A | 1.53 | 1.58 | 2.36 | 3.25 | 3.98 | 12.24 | 23.26 |
| Y708A/K709A | 0.92 | 1.31 | 1.55 | 1.51 | 3.76 | 6.35 | 16.72 |
| S387A/Q388A | 0.93 | 1.03 | 1.01 | 2.16 | 3.81 | 6.78 | 16.33 |
| N273A/T274A | 1.07 | 1.34 | 1.55 | 2.30 | 4.28 | 8.75 | 15.85 |
| N385A/G386A | 0.74 | 1.10 | 1.27 | 1.31 | 4.39 | 5.77 | 15.74 |
| T270A | 1.05 | 1.09 | 1.42 | 1.68 | 3.68 | 7.80 | 14.79 |
| W505A | 1.00 | 1.21 | 1.39 | 1.70 | 4.12 | 6.40 | 14.57 |
| T506A | 1.13 | 1.16 | 1.22 | 2.48 | 3.76 | 6.98 | 13.80 |
| L383A/N384A | 0.81 | 0.95 | 1.15 | 1.91 | 3.83 | 5.62 | 13.32 |
| N271A/D272A | 1.04 | 1.11 | 1.70 | 1.97 | 3.78 | 8.06 | 12.52 |
| P726A/R727A | 1.15 | 1.00 | 0.92 | 2.02 | 2.50 | 5.06 | 10.56 |
| T528A/H529A | 1.16 | 1.40 | 1.26 | 1.72 | 3.66 | 4.95 | 10.46 |
| G267A/G268A | 0.92 | 1.14 | 1.42 | 1.77 | 2.97 | 6.66 | 10.17 |

Table 6 shows AAV9 alanine mutations with slower blood clearance than the wild type. The listed mutations display retention in the blood at least 2 times higher than the wild-type AAV9 at 24 hours.

| Mutant | 1 m | 10 m | 30 m | 1 h | 4 h | 8 h | 24 h |
|---|---|---|---|---|---|---|---|
| AAV9 w/t | 1.00 | 1.00 | 1.00 | 1.00 | 1.00 | 1.00 | 1.00 |
| R585E | 0.92 | 0.93 | 1.11 | 1.24 | 1.41 | 1.94 | 4.51 |
| N262A/S263A | 1.09 | 1.00 | 1.16 | 1.37 | 1.85 | 2.16 | 5.16 |
| S268A/S269A | 1.04 | 1.04 | 1.16 | 1.31 | 1.63 | 2.13 | 5.03 |
| T264A/S265A | 1.03 | 1.19 | 1.14 | 1.34 | 1.67 | 2.14 | 5.03 |
| N272A | 1.12 | 1.07 | 1.14 | 1.35 | 1.84 | 2.03 | 4.49 |
| N270A/D271A | 0.94 | 1.06 | 1.13 | 1.18 | 1.48 | 1.99 | 4.36 |
| K258A/Q259A | 0.99 | 0.98 | 1.18 | 1.30 | 1.60 | 2.03 | 4.36 |
| G222A/S223A | 1.09 | 0.97 | 1.14 | 1.13 | 1.52 | 1.71 | 3.00 |
| N254A/H255A | 0.98 | 0.97 | 1.03 | 1.17 | 1.20 | 1.44 | 2.21 |
| I334A | 1.01 | 0.94 | 0.90 | 1.00 | 1.26 | 1.47 | 2.21 |

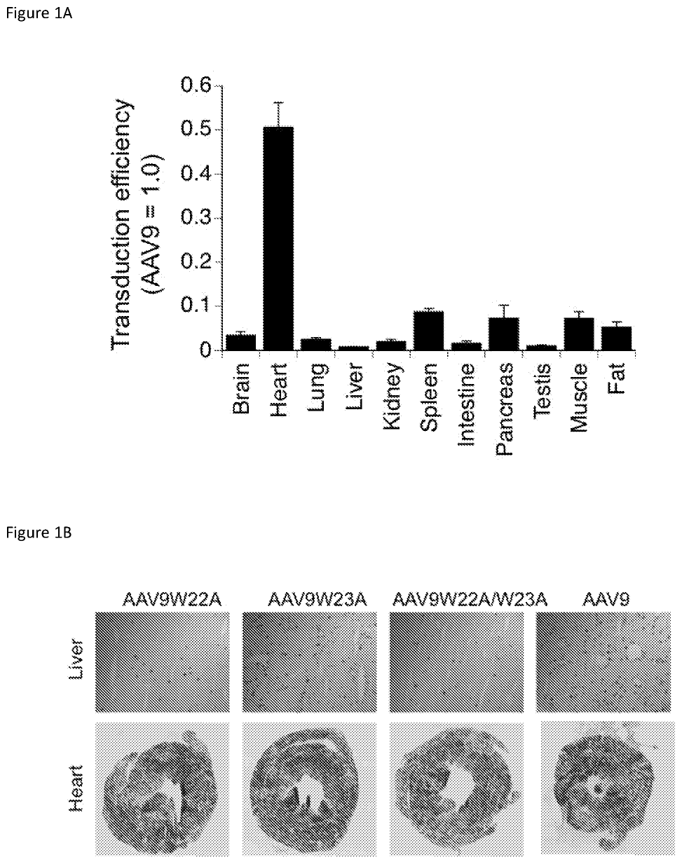

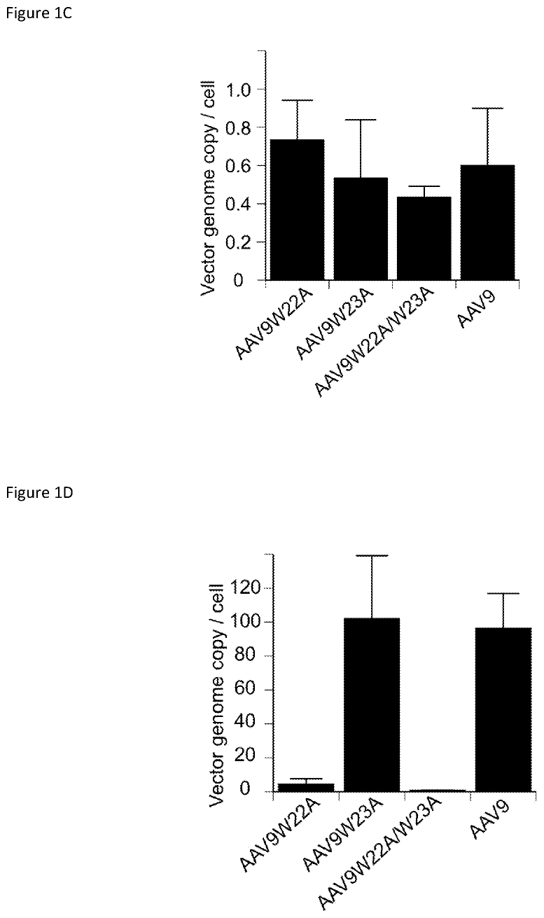

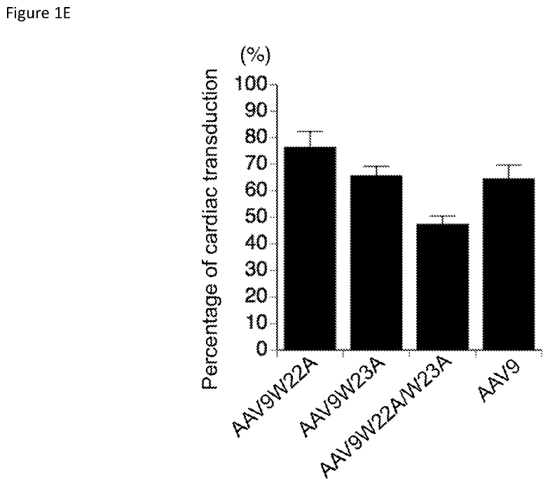

FIGS. 1A, 1B, 1C, 1D and 1E show liver-detargeting AAV9 mutants that transduce the heart at high levels. Single-stranded AAV-CMV-lacZ vectors packaged with 4 different capsids are indicated. Those capsids include AAV9W22A, AAV9W23A, AAV9W22A/W23A, and unmutated AAV9, and were injected into 8-week-old C57BL/6 male mice via the tail vein at a dose of 3×10¹¹ vg/mouse (n=3). The AAV-CMV-lacZ vector expresses the bacterial beta-galactosidase under the control of the human cytomegalovirus immediate early gene enhancer-promoter.

FIGS. 2A, 2B and 2C show liver-detargeting AAV9 mutants that transduce the brain at high levels. Double-stranded AAV-hSynl-GFP vectors packaged with 4 different capsids are indicated. Those capsids include AAV9N272A, AAV9-PHPB, AAV9N272A-PHPB, and unmutated AAV9 were injected into 8-week-old C57BL/6 male mice via the tail vein at a dose of 3×10¹¹ vg/mouse (n=2 to 3). The AAV-hSynl-lacZ vector expresses the enhanced green fluorescence protein (eGFP) under the control of the human synapsin I gene enhancer-promoter.

1. A recombinant adeno associated virus vector comprising a capsid protein of SEQ ID NO: 1 that has one or more of the mutations listed in Tables 1 and 5 herein.

2. The recombinant adeno associated virus vector of claim 1 comprising a capsid protein of SEQ ID NO: 1 that has one or more of the following single or double mutations: G28A, P30A/K31A, P32A/P33A, T265A/S266A, T270A, N271A/D272A, N273A/T274A, Y275A/F276A, G277A/Y278A, S387A/Q388A, W505A, T506A, T528A/H529A, and K530A/D531A, provided that the vector detargets the liver but transduces one or more of brain, heart, or skeletal muscle.

3. The recombinant viral vector of claim 1 comprising a capsid protein of SEQ ID NO: 1 that has one or more of the following single or double mutations: W10A/11A, G44A/L45A, V46A/L47A, P48A/G49A, Y50A/K51A, Y52A/L53A, G54A/P55A, F56A/N57A, G58A/L59A, D60A/K61A, D69A, E74A/H75A, D76A/K77A, Y79A, N88A/P89A, Y90A/L91A, R92A/Y93A, D97A, F100A/Q101A, V118A/F119A, K122A/K123A, K142A/K143A, R144A/P145A, R169A/K170A, R171A/L172A, N255A/H256A, K259A/Q260A, G267A/G268A, 1335A, L339A/T340A, L383A/N384A, N385A/G386A, G391A/R392A, D532A/E533A, S564A/E565A, E566A/E567A, T704A/S705A, N706A/Y707A, Y708A/K709A, 712A/V713A, V717A, P726A/R727A, provided that the vector detargets all of the brain, heart, lung, liver, kidney, spleen, intestine, pancreas, testis, muscle, or fat.

4. The recombinant viral vector of claim 3 further comprising an additional mutation that confers upon the vector an ability to transduce one or more tissues.

5. The recombinant viral vector of claim 1 comprising a capsid protein of SEQ ID NO: 1 that has one or both of the following double mutations: G86A/D87A or N94A/H95A provided that the vector transduces the liver but detargets all of the brain, heart, lung, liver, kidney, spleen, intestine, pancreas, testis, muscle or fat.

6. The recombinant viral vector of claim 1 comprising a capsid protein of SEQ ID NO: 1 that has one or more of the following double mutations: T265A/S266A, G267A/G268A, T270A, N271A/D272A, N273A/T274A, L383A/N384A, N385A/G386A, S387A/Q388A, W505A, T506A, T528A/H529A, Y708A/K709A, P726A/R727A, provided that the vector is retained at a concentration 10-fold higher in the blood following injection into an experimental animal than a viral vector comprising an unmutated capsid protein of SEQ ID NO: 1.

7. The recombinant viral vector of claim 1, comprising a capsid protein of SEQ ID NO: 3-63, or 107.

8. A recombinant viral vector comprising a capsid protein of SEQ ID NO: 2 that has one or more of the mutations listed in Tables 2, 3, and 4 herein.

9. The recombinant viral vector of claim 8 comprising a capsid protein of SEQ ID NO: 2 that has one or more of the following single or double mutations: W22A, W22A/W23A, K258A/Q259A, S268A/S269A, and N272A, provided that the vector detargets the liver but transduces one or more of brain, heart, or skeletal muscle.

10. The recombinant viral vector of claim 8 comprising a capsid protein of SEQ ID NO: 2 that has one or more of the following single or double mutations: W10A/11A, G44A/L45A, V46A/L47A, P48A/G49A, Y50A/K51A, Y52A/L53A, G54A/P55A, G56A/N57A, G58A/L59A, G62A/E63A, D69A, E74A/H75A, D76A/K77A, Y79A, Y90A/L91A, K92A/Y93A, D97A, F100A/Q101A, V118A/F119A, K122A/K123A, K142A/K143A, R144A/P145A, K168A/K169A, R170A/L171A, G222A/S223A, N254A/H255A, N262A/S263A, T264A/S265A, G266A/G267A, N270A/D271A, 1334A, and L338A/T339A provided that the vector detargets all of the brain, heart, lung, liver, kidney, spleen, intestine, pancreas, testis, muscle, or fat.

11. The recombinant viral vector of claim 10 where the capsid protein comprises an additional mutation that confers upon the vector an ability to transduce one or more tissues.

12. The recombinant viral vector of claim 8 comprising a capsid protein of SEQ ID NO: 2 that has one or both of the following double mutations: G86A/D87A or N94A/H95A provided that the vector transduces the liver but detargets all of the brain, heart, lung, liver, kidney, spleen, intestine, pancreas, testis, muscle or fat.

13. The recombinant viral vector of claim 8 comprising a capsid protein of SEQ ID NO:2 that has a N270A/D271A double mutation or an N272A mutation, provided that the vector binds CHO Pro5 cells but not to the surface of CHO Lec2 cells.

14. The recombinant viral vector of claim 8 comprising a capsid protein of SEQ ID NO: 2 that has one or more of the following double mutations: AAV9K258A/Q259A, AAV9N262A/S263A, AAV9T264A/S265A, AAV9G266A/G267A, and AAV9S268A/S269A provided that the vector is internalized in the cell but does not result in viral genome expression due to viral genome degradation.

15. The recombinant viral vector of claim 8 comprising a capsid protein of SEQ ID NO: 2 that has one or more of the following double mutations: AAV9W10A/L11A, AAV9G44A/L45A, AAV9V46A/L47A, AAV9P48A/G49A, AAV9Y50A/K51A, AAV9Y52A/L53A, AAV9G54A/P55A, AAV9G56A/N57A, AAV9G58A/L59A, AAV9D60A/K61A, AAV9G62A/E63A, AAV9N66A, AAV9D69A, AAV9E74A/H75A, AAV9D76A/K77A, AAV9Y79A, AAV9Q82A/L83A, AAV9G86A/D87A, AAV9N88A/P89A, AAV9Y90A/L91A, AAV9K92A/Y93A, AAV9N94A/H95A, AAV9D97A, AAV9F100A/Q101A, AAV9L104A/K105A, AAV9V118A/F119A, AAV9K122A/K123A, AAV9G222A/S223A, and AAV91334A, AAV9L338A/T339A, provided that the vector is internalized in the cell, is not degraded but does not result in viral genome expression.

16. The recombinant viral vector of claim 8 comprising a capsid protein of SEQ ID NO: 2 that has one or more of the following mutations: N262A/S263A, S268A/S269A, T264A/S265A, N272A, N270A/D271A, K258A/Q259A, G222A/S223A, N254A/H255A, 1334A provided that the vector is retained at a concentration 2-fold higher in the blood following injection into an experimental animal than a viral vector comprising an unmutated capsid protein of SEQ ID NO: 1.

17. The recombinant viral vector of claim 8 comprising a capsid protein of SEQ ID NO: 64-106 or 108-109.
